(19) United States
(12) Patent Application Publication
(10) Pub. No.: US 2010/0130721 A1
(43) Pub. Date: May 27, 2010

(54) IMMOBILIZED PROTEIN THAT IS IMMOBILIZED ONLY AT ITS AMINO TERMINUS IN ORIENTATION-CONTROLLED MANNER

(75) Inventors: Masahiro Iwakura, Ibaraki (JP); Kiyonori Hirota, Ibaraki (JP); Hiroyuki Sota, Ibaraki (JP); Gou Sarara, Ibaraki (JP); Hisashi Takahashi, Ibaraki (JP); Yukiko Aruga, Ibaraki (JP); Chiori Yamane, Ibaraki (JP)

Correspondence Address: FOLEY AND LARDNER LLP;SUITE 500, 3000 K STREET NW, WASHINGTON, DC, 20007, US

(73) Assignee: National Insititute of Advanced Industrial Science and Technology, Chiyoda-ku, Tokyo (JP)

(21) Appl. No.: 12/596,600

(22) PCT Filed: Apr. 17, 2008
(86) PCT No.: PCT/JP2008/057481

§ 371 Date: Oct. 19, 2009

(30) Foreign Application Priority Data
Apr. 20, 2007 (JP) 2007-112218

(51) Int. Cl. C07K 17/00

(52) U.S. Cl. 530/324; 530/402

(57) ABSTRACT

This invention provides an immobilized protein bound to an immobilization carrier at a protein amino terminus via the sole α-amino group of the protein comprising an amino acid sequence containing neither lysine residues nor cysteine residues represented by the general formula S1-R1-R2, wherein: the sequences are oriented from the amino terminal side to the carboxy terminal side; the sequence of the S1 portion may be absent, but when the sequence of the S1 portion is present, the sequence of the S1 portion is a spacer sequence composed of amino acid residues other than lysine and cysteine residues; the sequence of the R1 portion is the sequence of a subject protein to be immobilized and contains neither lysine residues nor cysteine residues; and the sequence of the R2 portion may be absent, but when the sequence of the R2 portion is present, the sequence of the R2 portion is a spacer sequence composed of amino acid residues other than lysine and cysteine residues.

### TECHNICAL FIELD

The present invention relates to immobilization of a protein containing neither lysine residues nor cysteine residues as amino acids that constitute a protein. A protein having such feature is useful for the preparation of an immobilized protein, and more particularly for the preparation of a protein that is immobilized in an orientation-controlled manner, for the preparation of a protein that is site-specifically and chemically modified, and for utilization thereof.

### BACKGROUND ART

A naturally occurring protein is composed of 20 types of amino acid residues; i.e., alanine, cysteine, aspartic acid, glutamic acid, phenylalanine, glycine, histidine, isoleucine, lysine, leucine, methionine, asparagine, proline, glutamine, arginine, serine, threonine, valine, tryptophane, and tyrosine. Properties of amino acid residues are influenced by properties of side-chain functional groups. When immobilization of a protein on an insoluble carrier is attempted with the utilization of side-chain reactivity, in general, the protein can be chemically bound to the carrier with the utilization of such reactivity. Examples of side-chain functional groups include the sulfhydryl group of cysteine, the ε-amino group of lysine (NH₂), and the carboxyl group of aspartic acid or glutamic acid. A fluorescent label or the like is introduced with the utilization of the reactivity of such functional groups.

A side-chain functional group of the cysteine residue, i.e., sulfhydryl, is a highly reactive amino acid residue that is extensively used for reactions such as S—S bonding, alkylation, or acylation. A side-chain functional group of the lysine residue, i.e., the ε-amino group (NH₂), has properties of a primary amine, which is an amino acid extensively used for reactions such as acetylation, alkylation, succinylation, or maleylation. An α-amino group exists at the protein amino terminus, and such amino group is known to have properties of a primary amine. The side-chain functional group of the aspartic acid or glutamic acid residue is a carboxyl group, and its reactivity is utilized in the same manner as the carboxyl group at the protein carboxy terminus. However, utilization thereof is less frequent than that of the aforementioned sulfhydryl group, ε-amino group (NH₂), or α-amino group. Under such circumstances, effective utilization of the reactivity of the sulfhydryl group or amino group that is a highly reactive functional group of a protein is considered to lead to extensive utilization of protein functions.

However, many naturally derived proteins generally comprise considerably over 100 amino acid residues. When an attention is paid to given amino acid residues, a plurality of such amino acid residues are present in each protein molecule. This disadvantageously complicates the control of the reaction when performing protein immobilization or chemical modification with the utilization of a functional group of a given amino acid. If an attention were to be paid to a given site of a protein sequence and a general technique for utilizing the chemical reactivity of its side-chain functional group can be developed, in particular, it is considered that this would result in the extensive utilization of proteins.

The present inventors have already prepared a protein in which a cysteine residue has been introduced into a sole protein C-terminal region, and they have converted a side-chain thiol group of the sole cysteine residue into a thiocyano group (i.e., conversion into a cyanocysteine group), thereby developing a method for orientation-controlled immobilization of a main chain (JP Patent No. 2990271, JP Patent No. 3047020, and JP Patent Publication (kokai) No. 2003-344396 A). They have developed a method for immobilizing and modifying a protein that is excellent in assured control of reaction homogenization, and they have demonstrated that such method is generally and extensively applicable to proteins. In the past, however, no method for assuring the certainty of the control of functional group reactivity was known except for the functional group of the cysteine residue. This hinders the more extensive utilization of proteins.

### DISCLOSURE OF THE INVENTION

### Objects to be Achieved by the Invention

An object of the present invention is to provide a general method for assuring the certainty of reactivity control of functional groups other than the cysteine residues. The present inventors have conducted concentrated studies in order to attain the above object. As a result, they discovered that preparation of a protein containing neither a cysteine residue nor a lysine residue would lead to assured control of the reactivity of the α-amino group that is the sole functional group of the protein. They verified their discovery with the use of several proteins and completed the present invention relating to an immobilized protein that is immobilized only at an amino terminus in an orientation-controlled manner. Similar effects can be expected when a protein containing no lysine residue is prepared; however, many functional groups having reactivity with amino groups are known to react with the SH group of the cysteine residue. Thus, the reactivity of the α-amino group can be completely controlled only when the sequence contains neither a cysteine residue nor a lysine residue.

### Means to Achieve the Object

The present inventors have already invented a protein to be used for immobilizing a portion of the protein represented by R1-R2 on an immobilization carrier, consisting of an amino acid sequence represented by the general formula R1-R2-R3-R4-R5, wherein:

the sequences are oriented from the amino terminal side to the carboxy terminal side;

the sequence of the R1 portion is the sequence of a subject protein to be immobilized and contains neither lysine residues nor cysteine residues;

the sequence of the R2 portion may be absent, but when the sequence of the R2 portion is present, the sequence of the R2 portion is a spacer sequence composed of amino acid residues other than lysine and cysteine residues;

the sequence of the R3 portion is composed of two amino acid residues represented by cysteine-X (where X denotes an amino acid residue other than lysine or cysteine);

the sequence of the R4 portion may be absent, but when the sequence of the R4 portion is present, the sequence of the R4 portion contains neither lysine residues nor cysteine residues, but contains an acidic amino acid residue capable of acidifying the isoelectric point of the entire protein consisting of the amino acid sequence represented by the general formula R1-R2-R3-R4-R5; and

the sequence of an R5 portion is an affinity tag sequence for protein purification. Further, the present inventors have demonstrated that the protein prepared by the present invention could assuredly control the reactivity of the functional group of the cysteine residue, and that a more homogeneous reaction product (i.e., R1-R2), which is a portion containing neither lysine residues nor cysteine residues, of the protein represented by the above general formula is cleaved from R3-R4-R5 by the reaction and is used for the immobilization reaction (JP Patent Application Nos. 2006-276468, 2007-057791, 2007-059175, and 2007-059204).

Further, the present inventors have studied a portion containing neither lysine residues nor cysteine residues (i.e., R1-R2). In such sequence, an α-amino group as the amino terminus is the sole amino group, and utilization thereof as a functional group can secure the control of reactivity of the functional group. Also, an example of the usefulness of such sequence is the applicability thereof for production of a protein immobilized in an orientation-controlled manner at the protein amino terminus. When preparing a portion containing neither lysine residues nor cysteine residues (i.e., R1-R2), a protein represented by the general formula R1-R2-R3-R4-R5 is used as a starting material, the sole cysteine residue therein is converted into the cyano group, and a peptide chain cleavage reaction is carried out with the utilization of reactivity of cyanocysteine to divide the sequence into the R1-R2 portion and the R3-R4-R5 portion. Thus, a sequence of interest can be generated.

As a result, the present inventors newly developed a protein comprising the amino acid sequence represented by the general formula S1-R1-R2, wherein:

the sequences are oriented from the amino terminal side to the carboxy terminal side;

the sequence of the S1 portion may be absent, but when the sequence of the S1 portion is present, the sequence of the S1 portion is a spacer sequence composed of amino acid residues other than lysine and cysteine residues;

the sequence of the R1 portion is the sequence of a subject protein to be immobilized and contains neither lysine residues nor cysteine residues; and

the sequence of the R2 portion may be absent, but when the sequence of the R2 portion is present, the sequence of the R2 portion is a spacer sequence composed of amino acid residues other than lysine and cysteine residues as an orientation-controlled immobilized protein, thereby completing the present invention.

Specifically, the embodiments of the present invention are as follows.

(1) An immobilized protein bound to an immobilization carrier at a protein amino terminus via the sole α-amino group of the protein consisting of an amino acid sequence containing neither lysine residues nor cysteine residues represented by the general formula S1-R1-R2, wherein:

the sequences are oriented from the amino terminal side to the carboxy terminal side;

the sequence of the S1 portion may be absent, but when the sequence of the S1 portion is present, the sequence of the S1 portion is a spacer sequence composed of amino acid residues other than lysine and cysteine residues;

the sequence of the R1 portion is the sequence of a subject protein to be immobilized and contains neither lysine residues nor cysteine residues; and

the sequence of the R2 portion may be absent, but when the sequence of the R2 portion is present, the sequence of the R2 portion is a spacer sequence composed of amino acid residues other than lysine and cysteine residues.

(2) An immobilized protein consisting of the amino acid sequence represented by the general formula S1-R1-R2, wherein, in the amino acid sequence of the general formula S1-R1-R2, the sequence of the R1 portion is:

the sequence remaining unchanged when the amino acid sequence of a naturally derived protein contains neither lysine residues nor cysteine residues; or

the amino acid sequence of a protein that consists of an amino acid sequence modified to contain neither lysine residues nor cysteine residues and has functions equivalent to those of a naturally derived protein in which a modified amino acid sequence is obtained by substituting all lysine and cysteine residues in the amino acid sequence with amino acid residues other than lysine and cysteine residues, when the sequence contains lysine residues and cysteine residues.

(3) The immobilized protein according to (1) or (2) wherein, in the amino acid sequence of the general formula S1-R1-R2, the sequence of the R1 portion has a function of interacting specifically with an antibody molecule.

(4) The immobilized protein wherein, in the amino acid sequence represented by the general formula S1-R1-R2,
S1=Ser-Gly-Gly-Gly-Gly or is absent,
R1=(Ala-Asp-Asn-Asn-Phe-Asn-Arg-Glu-Gln-Gln- Asn-Ala-Phe-Tyr-Glu-Ile-Leu-Asn-Met-Pro- Asn-Leu-Asn-Glu-Glu-Gln-Arg-Asn-Gly-Phe- Ile-Gln-Ser-Leu-Arg-Asp-Asp-Pro-Ser-Gln- Ser-Ala-Asn-Leu-Leu-Ser-Glu-Ala-Arg-Arg- Leu-Asn-Glu-Ser-Gln-Ala-Pro-Gly)n (where n is an arbitrary integer ranging from 1 to 5), and
R2=Gly-Gly-Gly-Gly or is absent.

(5) The immobilized protein wherein, in the amino acid sequence represented by the general formula S1-R1-R2,
S1=absent;
R1=(Ala-Tyr-Arg-Leu-Ile-Leu-Asn-Gly-Arg-Thr- Leu-Arg-Gly-Glu-Thr-Thr-Thr-Glu-Ala-Val- Asp-Ala-Ala-Thr-Ala-Glu-Arg-Val-Phe-Arg- Gln-Tyr-Ala-Asn-Asp-Asn-Gly-Val-Asp-Gly- Glu-Trp-Thr-Tyr-Asp-Asp-Ala-Thr-Arg-Thr- Phe-Thr-Val-Thr-Glu-Arg-Pro-Glu-Val-Ile- Asp-Ala-Ser-Glu-Leu-Thr-Pro-Ala-Val-Thr-Pro-Gly)n (where n is an arbitrary integer ranging from 1 to 5), and
R2=Gly-Gly-Gly-Gly or is absent.

(6) A carrier on which the immobilized proteins according to any of (1) to (5) are immobilized.

### EFFECTS OF THE INVENTION

With the utilization of the protein of the present invention, reactivity of functional groups, such as in the case of immobilization of the protein or introduction of a fluorescent group, can be assuredly controlled with the use of a sole amino group; i.e., the α-amino group. When immobilizing a protein, in particular, a protein can be immobilized on a main chain at a single site mediated by the protein α-amino group, which enables orientation-controlled immobilization of the protein. The present invention is based on the assumption that a sequence containing neither lysine residues nor cysteine residues as R1 can be obtained; however, it is obvious to a person skilled in the art that utilization of currently available findings and techniques would be sufficient to obtain such sequence and that there is no technical restriction. Thus, the present invention is generally applicable.

### PREFERRED EMBODIMENTS OF THE INVENTION

The present invention will be described in detail as follows.

The term “protein” used in the present invention refers to a protein that is expressed as a protein comprising the amino acid sequence represented by the general formula S1-R1-R2. In such general formula, the sequence is an amino acid sequence oriented from the amino terminal side to the carboxy terminal side. The sequence of the S1 portion may be absent, but when the sequence of the S1 portion is present, the sequence of the S1 portion is a spacer sequence composed of amino acid residues other than lysine and cysteine residues, the sequence of the R1 portion is a protein sequence for exhibiting desired functions, such as functions for binding or catalytic functions, which contains neither a lysine residue or cysteine residue, and the sequence of the R2 portion may be absent, but when the sequence of the R2 portion is present, the sequence of the R2 portion is a spacer sequence composed of amino acid residues other than lysine and cysteine residues.

In the case of the present invention, the R1 portion is responsible for target functions. When immobilization of the protein of the present invention is to be mediated by the amino terminal α-amino group, the spacer sequence of the S1 portion is occasionally necessary in order to maximize the functions of the R1 portion. When the protein of the present invention is expressed and purified via tag purification, the spacer sequence of the R2 portion occasionally becomes effective for cleaving the tag sequence used for purification. In such a case, the protein of the present invention is used as a sequence comprising the R2 sequence added thereto. In the R1 portion, further, a sequence unit exerting desired functions may be repeated to enhance the functions. The sequence of the R1 portion can be designed based on a naturally derived protein sequence. Naturally derived proteins are generally composed of 20 types of amino acid residues including lysine and cysteine residues. In such a case, the lysine residue and the cysteine residue should be substituted with any one of 18 types of amino acid other than lysine or cysteine such that the resultant can retain the functions of the original natural protein.

The present inventors have already established methods for preparing proteins containing neither cysteine nor methionine (JP Patent Republication No. 01/000797, M. Iwakura et al. J. Biol. Chem. 281, 13234-13246 (2006), JP Patent Publication (Kokai) No. 2005-058059 A). With the use of a method similar to these methods, a protein comprising an amino acid sequence composed of 18 types of amino acid containing neither a cysteine residue nor a lysine residue and exerting functions equivalent to those of a natural protein can be prepared by amino acid sequence conversion based on the amino acid sequence of the naturally derived protein. The outline of this method is as described below.

1. All cysteine residue portions and lysine residue portions in a natural sequence are subjected to comprehensive single amino acid substitution and then the functions are examined.

2. Mutants obtained via single amino acid substitution of each residue portion are ranked in order of desirability of functions. The mutations of the top three mutants excluding substitutions with cysteine or lysine are carried out in combination. The mutations of the top three mutants are selected again and carried out in combination with the mutations of the top three mutants obtained via single amino acid substitutions of the other sites (excluding substitutions with cysteine or lysine).

3. This procedure is repeated until all cysteine residue portions and lysine residue portions are substituted with other amino acids.

More specifically, the procedure is carried out as follows.

It is assumed that there are “n (number)” lysine and cysteine residues in a natural protein with a full-length of “m (number)” amino acids. The position of each residue on the amino acid sequence is determined to be Ai (i=1 to n).

The thus obtained mutation is represented by A1/MA1.

Regarding lysine and cysteine residues represented by Ai (i=2 to n) at other sites, a mutant gene is prepared by substituting codons encoding lysine and cysteine residues with codons encoding the above “amino acids other than lysine or cysteine” (maximum 18 types). The mutant gene is expressed and then the enzyme activity of the thus obtained double mutant enzyme protein is examined.

When the activity of the double mutants is examined, mutants exhibiting activity equivalent to or higher than that of the natural protein are observed. Up to three double mutants are selected from the double mutants in decreasing order of activity.

Next, triple mutants (maximum 3×18=54 types) are prepared by substituting lysine and cysteine residues of A3 of each of the thus obtained double mutants with amino acids (maximum 18 types) other than lysine and cysteine residues. The enzyme activity is then examined.

When the activity of triple mutants is examined, mutants exhibiting activity equivalent to or higher than that of the natural protein are observed.

Hereinafter, fourfold, ••, n-fold mutants are prepared similarly. The final n-fold mutant is a target protein containing neither lysine residues nor cysteine residues.

With this procedure, a protein at least having functions equivalent to those of the original natural protein can be obtained. The phrase “functions equivalent to those of the original natural protein” means that the activity of the protein obtained via sequence modification remains unchanged in terms of quality and is not lowered significantly in terms of amount compared with the original natural protein. For example, when an original natural protein is an enzyme that catalyzes a specific reaction, the protein obtained via sequence modification also has enzyme activity that catalyzes the same reaction. Alternatively, when an original natural protein is an antibody that binds to a specific antigen, the protein obtained via sequence modification has activity of an antibody capable of binding to the same antigen. The activity of a protein obtained via amino acid sequence modification accounts for 10% or more, preferably 50% or more, more preferably 75% or more, and particularly preferably 100% or more of the activity of the original natural protein. In the case of an enzyme, activity is represented by specific activity, for example. In the case of a protein capable of binding to another substance such as an antibody, activity is represented by binding ability. Methods for measuring such activity can be adequately selected depending on proteins.

The present inventors have already demonstrated that, when partial sequences of different natural proteins capable of binding to antibody molecules are converted to sequences containing neither a cysteine residue nor a lysine residue, the converted partial sequences have functions equivalent to those of the partial sequence derived from natural proteins (JP Patent Application Nos. 2006-276468, 2007-057791, 2007-059175, and 2007-059204). For example, domain A of Staphylococcus-derived protein A (SEQ ID NOs: 1 and 2), domain G1 of Streptococcus-derived protein G (SEQ ID NOs: 3 and 4), and domain B of Peptostreptococcus-derived protein L (SEQ ID NOs: 5 and 6) have been demonstrated. This indicates the presence of a protein that comprises an amino acid sequence modified to be composed of 18 types of amino acid containing neither a cysteine residue nor a lysine residue based on the amino acid sequence of a natural protein having specific functions and retains functions equivalent to those of the naturally existing protein. This also suggests the universality of the present invention such that the present invention is applicable to all proteins. Also, it is predicted that a protein having target functions can be prepared by a de novo design technique or the like that involves artificially designing such a protein from an amino acid sequence and then synthesizing the protein. It is also suggested herein that a functional protein can be prepared via limitation such that 18 types of amino acid alone (containing neither a cysteine residue nor a lysine residue) are used in the de novo design technique, for example. It is also suggested herein that not only modification of the amino acid sequence of a naturally derived protein, but also design and preparation of a novel functional protein having specific functions, which can be used as the R2 portion of the present invention, are possible.

Examples of the protein of the R1 portion include a protein having enzyme activity and a protein capable of binding to an antibody molecule. Known examples of a protein capable of binding to an antibody molecule include protein A derived from Staphylococcus aureus (disclosed in A. Forsgren and J. Sjoquist, J. Immunol. (1966) 97, 822-827), protein G derived from Streptococcus sp. Group C/G (disclosed in the specification of EP Application (published) No. 0131142A2 (1983)), protein L derived from Peptostreptococcus magnus (disclosed in the specification of U.S. Pat. No. 5,965,390 (1992)), protein H derived from group A Streptococcus (disclosed in the specification of U.S. Pat. No. 5,180,810 (1993)), protein D derived from Haemophilus influenzae (disclosed in the specification of U.S. Pat. No. 6,025,484 (1990)), protein Arp (Protein Arp4) derived from Streptococcus AP4 (disclosed in the specification of U.S. Pat. No. 5,210,183 (1987)), Streptococcal FcRc derived from group C Streptococcus (disclosed in the specification of U.S. Pat. No. 4,900,660 (1985)), a protein derived from group A streptococcus, Type II strain (disclosed in U.S. Pat. No. 5,556,944 (1991)), a protein derived from Human Colonic Mucosal Epithelial Cell (disclosed in the specification of U.S. Pat. No. 6,271,362 (1994)), a protein derived from Staphylococcus aureus, strain 8325-4 (disclosed in the specification of U.S. Pat. No. 6,548,639 (1997)), and a protein derived from Pseudomonas maltophilia (disclosed in the specification of U.S. Pat. No. 5,245,016 (1991)).

Based on the sequences of naturally derived proteins having such functions or domains exerting functions of such proteins, sequences containing no cysteine or lysine can be produced while maintaining the functions.

Through modification of the sequence (SEQ ID NO: 6) derived from domain A of Staphylococcus-derived protein A as shown below, for example,

Ala-Asp-Asn-Asn-Phe-Asn-Lys-Glu-Gln-Gln-Asn-Ala-
Phe-Tyr-Glu-Ile-Leu-Asn-Met-Pro-Asn-Leu-Asn-Glu-
Glu-Gln-Arg-Asn-Gly-Phe-Ile-Gln-Ser-Leu-Lys-Asp-
Asp-Pro-Ser-Gln-Ser-Ala-Asn-Leu-Leu-Ser-Glu-Ala-
lys-lys-Leu-Asn-Glu-Ser-Gln-Ala-Pro-Lys

the sequence (SEQ ID NO: 7) as shown below

Ala-Asp-Asn-Asn-Phe-Asn-Arg-Glu-Gln-Gln-Asn-Ala-
Phe-Tyr-Glu-Ile-Leu-Asn-Met-Pro-Asn-Leu-Asn-Glu-
Glu-Gln-Arg-Asn-Gly-Phe-Ile-Gln-Ser-Leu-Arg-Asp-
Asp-Pro-Ser-Gln-Ser-Ala-Asn-Leu-Leu-Ser-Glu-Ala-
Arg-Arg-Leu-Asn-Glu-Ser-Gln-Ala-Pro-Gly

can be obtained as the sequence of a protein containing neither a cysteine residue nor a lysine residue and having IgG binding activity equivalent to that of the naturally derived protein comprising the above sequence (SEQ ID NO: 6). Many mutants obtained via amino acid substitution with amino acids other than cysteine or lysine in the above sequence exhibit IgG binding activity. A sequence comprising a repeat of this sequence also exhibits IgG binding activity.

Through modification of the sequence (SEQ ID NO: 8) derived from domain G1 of Streptococcus-derived protein G as shown below

Thr-Tyr-Lys-Leu-Ile-Leu-Asn-Gly-Lys-Thr-
Leu-Lys-Gly-Glu-Thr-Thr-Thr-Glu-Ala-Val-
Asp-Ala-Ala-Thr-Ala-Glu-Lys-Val-Phe-Lys-
Gln-Tyr-Ala-Asn-Asp-Asn-Gly-Val-Asp-Gly-
Glu-Trp-Thr-Tyr-Asp-Asp-Ala-Thr-Lys-Thr-
Phe-Thr-Val-Thr-Glu-Arg-Pro-Glu-Val-Ile-
Asp-Ala-Ser-Glu-Leu-Thr-Pro-Ala-Val-Thr

the sequence (SEQ ID NO: 9) as shown below

Ala-Tyr-Arg-Leu-Ile-Leu-Asn-Gly-Arg-Thr-Leu-Arg-
Gly-Glu-Thr-Thr-Thr-Glu-Ala-Val-Asp-Ala-Ala-Thr-
Ala-Glu-Arg-Val-Phe-Arg-Gln-Tyr-Ala-Asn-Asp-Asn-
Gly-Val-Asp-Gly-Glu-Trp-Thr-Tyr-Asp-Asp-Ala-Thr-
Arg-Thr-Phe-Thr-Val-Thr-Glu-Arg-Pro-Glu-Val-Ile-
Asp-Ala-Ser-Glu-Leu-Thr-Pro-Ala-Val-Thr-Pro-Gly

can be obtained as the sequence of a protein containing neither a cysteine residue nor a lysine residue and having IgG binding activity equivalent to that of the naturally derived protein comprising the above sequence (SEQ ID NO: 8). Many mutants obtained via amino acid substitution with amino acids other than cysteine or lysine in the above sequence exhibit IgG binding activity. A sequence comprising a repeat of this sequence also exhibits IgG binding activity.

Further, through modification of the sequence (SEQ ID NO: 10) derived from domain B1 of Peptostreptococcus-derived protein L as shown below

Val-Thr-Ile-Lys-Ala-Asn-Leu-Ile-Tyr-Ala-
Asp-Gly-Lys-Thr-Gln-Thr-Ala-GIu-Phe-Lys-
Gly-Thr-Phe-Glu-Glu-Ala-Thr-Ala-Glu-Ala-
Tyr-Arg-Tyr-Ala-Asp-Leu-Leu-Ala-Lys-Glu-
Asn-Gly-Lys-Tyr-Thr-Val-Asp-Val-Ala-Asp-
Lys-Gly-Tyr-Thr-Leu-Asn-Ile-Lys-Phe-Ala

the sequence (SEQ ID NO: 11) as shown below

Ala-Thr-Ile-Arg-Ala-Asn-Leu-Ile-Tyr-Ala-Asp-Gly-
Arg-Thr-Gln-Thr-Ala-Glu-Phe-Arg-Gly-Thr-Phe-Glu-
Glu-Ala-Thr-Ala-Glu-Ala-Tyr-Arg-Tyr-Ala-Asp-Leu-
Leu-Ala-Arg-Glu-Asn-Gly-Arg-Tyr-Thr-Val-Asp-Val-
Ala-Asp-Arg-Gly-Tyr-Thr-Leu-Asn-Ile-Arg-Phe-Ala-
Pro-Gly

can be obtained as the sequence of a protein containing neither a cysteine residue nor a lysine residue and having IgG binding activity equivalent to that of the naturally derived protein comprising the above sequence (SEQ ID NO: 10). Many mutants obtained via amino acid substitution with amino acids other than cysteine or lysine in the above sequence exhibit IgG binding activity. A sequence comprising a repeat of this sequence also exhibits IgG binding activity.

When the sequence represented by R1 is repeated, the number of repetition is not limited, and such number is 2 to 10, and preferably 2 to 5, for example.

By introducing an adequate spacer sequence into an amino terminal or carboxy terminal side of the above sequence, convenience of the use thereof can be improved while maintaining functions of a protein containing neither a cysteine residue nor a lysine residue.

When immobilizing the protein by introducing an adequate spacer sequence represented by the general formula 51 into an amino terminal side, for example, the protein may be immobilized while maintaining an adequate distance from the immobilization base material to minimize the influence by the immobilization base material. The S1 sequence may be any sequence, provided that such sequence is composed of amino acids other than cysteine or lysine. In view of the role as a linker, it is obvious that the S1 sequence that can independently exert functions, such as binding activity or catalytic activity, is not the target sequence. The simplest spacer sequence is a chain of glycine. A specific example thereof is polyglycine comprising 0 to 10 or 2 to 5 glycines, such as Gly-Gly-Gly-Gly (SEQ ID NO: 3). When such effects cannot be attained at significant levels, it is obvious that introduction of a spacer sequence is not necessary.

When a fusion protein are to be expressed and produced by introducing an adequate spacer sequence represented by the general formula R2 into a carboxy terminal side, the introduced purification tag can be efficiently removed. The R2 sequence may be any sequence, provided that such sequence is composed of amino acids other than cysteine or lysine. In view of the role as a linker, it is obvious that the R2 sequence that can independently exert functions, such as binding activity or catalytic activity, is not the target sequence. The simplest spacer sequence is a chain of glycine. A specific example thereof is polyglycine comprising 0 to 10 or 2 to 5 glycines, such as Gly-Gly-Gly-Gly (SEQ ID NO: 3). When such effects cannot be attained at significant levels, it is obvious that introduction of a spacer sequence is not necessary.

The protein comprising the amino acid sequence represented by the general formula S1-R1-R2 of the present invention can be prepared by a so-called recombinant DNA technique. Such protein can be chemically synthesized in accordance with a sequence. When the protein is prepared via the recombinant DNA technique, for example, a codon is adequately selected in accordance with the sequence, the start codon and the stop codon are added, the SD sequence required for initiation of translation and a promoter sequence required for initiation of transcription are operably linked and introduced into sites upstream of the start codon, the gene as the expression unit is synthesized, the resultant is introduced into an adequate plasmid or the like, the resultant is transduced into a host cell to prepare an expression cell, the resultant is cultured, and the target protein is adequately separated and purified from the culture resulting from expression and accumulation of protein in the host cell. Thus, a homogeneous sample can be obtained. A person skilled in the art can implement such procedure without particular difficulty.

When the protein is prepared by a so-called recombinant DNA technique, it is suggested that a tag sequence is used in order to more efficiently separate and purify the protein.

An example of a tag sequence is a sequence that can bind to a specific compound; i.e., an affinity tag sequence. When a protein containing the aforementioned tag is purified with the use of an antibody specific for such tag, an epitope tag may be used. An example of such an affinity tag sequence is a polyhistidine sequence comprising 2 to 12, preferably 4 or more, more preferably 4 to 7, and further preferably 5 or 6 histidines. In this case, the above polypeptide can be purified by nickel chelate column chromatography using nickel as a ligand. Also, the polypeptide can be purified by affinity chromatography using a column to which an antibody against polyhistidine has been immobilized as a ligand. In addition to such tags, a HAT tag, a HN tag, and the like comprising histidine-containing sequences can also be used. Examples of tags and ligands to be used for affinity chromatography are as listed below, but the examples are not limited thereto. All known affinity tags (epitope tags) can be used herein. Other examples of affinity tags include a V5 tag, an Xpress tag, an AU1 tag, a T7 tag, a VSV-G tag, a DDDDK tag, an S tag, CruzTag09, CruzTag 22, CruzTag41, a Glu-Glu tag, a Ha.11 tag, and a KT3 tag.

| | |
|---|---|
| Tag | ligand |
| Glutathione-S-transferase (GST) | glutathione |
| Maltose binding protein (MBP) | amylase |
| HQ tag (HQHQHQ; SEQ ID NO: 12) | nickel |
| Myc tag (EQKLISEEDL; SEQ ID NO: 13) | anti-Myc |
| | antibody |
| HA tag (YPYDVPDYA; SEQ ID NO: 14) | anti-HA |
| | antibody |
| FLAG tag (DYKDDDDK; SEQ ID NO: 15) | anti-FLAG |
| | antibody |

When a tag sequence for purification is used, it is required that the protein is expressed as a fusion protein of the tag sequence (it is referred to as “T1”) and the sequence represented by the general formula S1-R1-R2 of the present invention, the protein is separated and purified, and the tag sequence portion is adequately removed. To this end, it is necessary to introduce a cleavage sequence (it is referred to as “C1”), which enables specific cleavage, into a site between the tag sequence and the sequence represented by the general formula S1-R1-R2. To this end, fusion protein sequences are classified as two types of sequences shown below.

1: general formula T1-C1-S1-R1-R2 (type 1 fusion protein)

2: general formula S1-R1-R2-C1-T1 (type 2 fusion protein)

The amino acid sequence represented by the general formula S1-R1-R2 of the present invention is characterized in that the sequence contains neither the cysteine nor lysine residue. This enables the use of common sequences for specific cleavage.

In the case of the type 1 protein, a lysine residue may be used as the C1 sequence to treat the carboxy terminal side of the sole lysine residue of the type 1 fusion protein with lysyl endopeptidase, so that the T1-C1 portion can be separated from the S1-R1-R2 portion. In the present invention, an example of the “sequence” is a sequence consisting of a single amino acid.

In the case of the type 2 fusion protein, an amino acid sequence comprising 2 amino acids represented by cysteine-X (where X denotes an amino acid other than lysine or cysteine) can be used as the C1 sequence. With the use of this sequence, the sole cysteine in the type 2 fusion protein is subjected to cyanation, and the cleavage reaction utilizing the reactivity of cyanocysteine is performed, so that the reaction of the S1-R1-R2 portion can be more effectively carried out.

The cleavage reaction involving cyanocysteine is represented by the reaction formula NH₂—R—CO—NH—CH(CH₂—SCN)—CO—X+H₂O→NH₂—R—COOH+ITC-CO—X wherein R denotes an arbitrary amino acid sequence, X denotes OH, an arbitrary amino acid, or an arbitrary amino acid sequence, and ITC denotes 2-imidazolidene-4-carboxyl group. In general, a method involving the use of a cyanation reagent for the reaction, such as 2-nitro-5-thiocyanobenzoic acid (VTCB) (see Y. Degani, A. Ptchornik, Biochemistry, 13, 1-11 (1974)) or 1-cyano-4-dimethylaminopyridinium tetrafluoroborate (CDAP), is convenient. Commercially available NTCB and CDAP can be used without modification. Cyanation with the use of NTCB can be efficiently carried out at a pH level ranging from 7 to 9, and the reaction efficiency can be inspected based on an increase in the absorbance of free thionitrobenzoic acid at 412 nm (molecular extinction coefficient=13,600 M-1 cm-1). The SH group can be cyanated in accordance with the method described in the document (J. Wood & Catsipoolas, J. Biol. Chem. 233, 2887 (1963)).

After the cleavage reaction, the S1-R1-R2 portion can be separated from the C1-T1 portion and purified with the use of an affinity carrier used for purifying the tag sequence represented by T1. This can facilitate recovery of a protein that does not bind to the affinity carrier.

An example of a form of the use of a protein comprising the amino acid sequence represented by the general formula S1-R1-R2 of the present invention is orientation-controlled immobilization thereof to an immobilization carrier. Immobilization involves the use of the properties of the sole α-amino group in the protein as the primary amine as a functional group. In order to perform immobilization, it is necessary to activate a carrier and perform a chemical reaction. Combinations of a functional group of a carrier and a method for activating the same are as follows.

Counterpart functional group: hydroxyl group (OH)-activation method: cyanogen bromide method

Counterpart functional group: hydroxyl group (OH)-activation method: epoxy method

Counterpart functional group: hydroxyl group (OH)-activation method: oxysilane method

Counterpart functional group: a carboxyl group (COOH)-activation method: carbodiimide method

Counterpart functional group: amide group (CONH₂)-activation method: glutaraldehyde method

Counterpart functional group: amide group (CONH₂)-activation method: hydrazine (acyl azide) method

As carrier base materials that can be used with such combinations, silica, glass, plastic materials represented by polyethylene, polypropylene, or polystyrene, hydrogel, and the like can be extensively used. Examples of “carrier” in the present invention include any carriers such as particulate carriers, monolith carriers, and plate-like or sheet-like base materials, as long as they are insoluble and proteins can be immobilized thereon. Examples of an “immobilization carrier” include “immobilization base materials.” Moreover, an “immobilization carrier” may also be referred to as an “insoluble carrier.” Examples of a commercially available carrier having an amide group include Amino-Cellulofine (commercially available from Seikagaku Corporation), AF-Amino Toyopearl (marketed by TOSOH), EAH-Sepharose 4B and Lysine-Sepharose 4B (commercially available from Amersham Biosciences), Porus 20NH (commercially available from Boehringer Mannheim), CNBr-activated Sepharose FF, and NHS-activated Sepharose FF. Also, a primary amino group is introduced onto glass beads, glass plates, or the like using a silane compound (e.g., 3-aminopropylmethoxysilane) that has a primary amino group and then the resultant can also be used.

Some of these activation methods involve the use of strong alkaline reagents or active drugs; however, such agents are used when activating solids or semi-solids alone, and the reaction is allowed to proceed by introducing a protein under mild conditions after the completion of activation. Thus, it would not raise any problem. The present invention is advantageous in that the reaction can be carried out without imposing burdens on proteins.

The protein of the present invention can be immobilized on a carrier at a single amino terminal site of the protein in an orientation-controlled manner.

The present invention provides an immobilized protein comprising an amino acid sequence containing neither the cysteine residue nor the lysine residue obtained by the above method, which is bound to an immobilization carrier mediated by an adequate linker sequence, and a carrier on which the immobilized protein has been immobilized.

### EXAMPLES

The present invention will be described in detail by examples as follows, but the present invention is not limited by these examples.

In the following Examples, experimental methods described below were used commonly.

### [Gene Synthesis]

As proteins to be expressed by synthetic genes, all genes were designed so as to be expressed in the form of the aforementioned type 2 fusion protein (a protein having the sequence represented by the general formula S1-R1-R2-C1-T1). In such a case, Cys-Ala was used as a common sequence of the C1 amino acid sequence, and Asp-Asp-Asp-Asp-Asp-Asp-His-His-His-His-His-His (SEQ ID NO: 16) was used as a common sequence of the T1 amino acid sequence. As properties of the tag of T1, properties as a His-tag were utilized and designed so as to be capable of affinity purification with a nickel chelate column.

Genes described in the Examples were synthesized by contracted manufacturers of synthetic genes, unless otherwise specified. dsDNA was synthesized based on a nucleotide sequence shown in each case and then inserted into the BamHI-EcoRI site of a pUC18 vector. The sequences of the thus obtained clones were confirmed by single strand analysis and then the nucleotide sequence information was verified. Sites for which mismatches had been confirmed were subjected to correction using a technique such as site directed mutagenesis, and then the thus obtained plasmid DNA (approximately 1 microgram) was introduced. Regarding the target portion in the plasmid introduced, the sequence was confirmed again by sequencing.

### [Preparation of Mutant by Single Amino Acid Substitution]

Amino acid substitution was carried out according to a QuickChange method (described for a QuickChange Site-Directed Mutagenesis kit, Stratagene) using a DNA primer prepared by converting a DNA sequence encoding an amino acid at a substitution site to a target codon sequence so that 24 bases of the original sequence were present on both of its ends and its complementary DNA primer.

### [Measurement of Protein Concentration]

Protein concentration was determined by assaying the absorbance at 224 nm and 233.3 nm, unless otherwise specified (W. E. Groves, et al., Anal. Biochem., 22, 195-210 (1968)).

### [Purification of Fusion Protein]

Escherichia coli JM109 strain transformed with a recombinant plasmid was cultured overnight at 35° C. in 2 liters of medium (containing 20 g of sodium chloride, 20 g of yeast extract, 32 g of tryptone, and 100 mg of ampicillin sodium). Subsequently, the culture solution was centrifuged at a low speed (5,000 rotations per minute) for 20 minutes, so that 3 g to 5 g of cells (wet weight) was obtained. This was suspended in 20 ml of 10 mM phosphate buffer (pH 7.0). The cells were disrupted with a French press and then centrifuged at a high speed for 20 minutes (20,000 rotations per minute), so that a supernatant was separated. Streptomycin sulfate was added to the thus obtained supernatant to a final concentration of 2%. After 20 minutes of stirring, the solution was centrifuged at a high speed (20,000 rotations per minute) for 20 minutes, so that a supernatant was separated. Subsequently, ammonium sulfate treatment was carried out. The thus obtained supernatant was applied to a nickel chelate column (purchased from GE Healthcare Biosciences). The column was sufficiently washed using 200 ml or more of washing buffer (5 mM imidazole, 20 mM sodium phosphate, 0.5 M sodium chloride; pH 7.4). After washing, 20 ml of elution buffer (0.5 M imidazole, 20 mM sodium phosphate, 0.5 M sodium chloride; pH 7.4) was applied, so that a target protein was eluted. Subsequently, to remove imidazole from the protein solution, dialysis was carried out against 5 liters of 10 mM phosphate buffer (pH 7.0). MWCO3500 (purchased from Spectrum Laboratories) was used as a dialysis membrane. After dialysis, the target protein was dried using a centrifugal vacuum dryer.

### [Analysis of Binding Properties to Human Antibody IgG Molecule]

A Biacore surface plasmon resonance biosensor (Biacore) was used for analyzing the binding properties of target proteins, and the analysis was carried out according to protocols provided by Biacore. Running buffer with a composition of 10 mM HEPES (pH 7.4), 150 mM sodium chloride, 5 μM EDTA, and 0.005% Surfactant P20 (Biacore), which had been deaerated in advance, was used. As a sensor chip, a Sensor Chip NTA (Biacore) was used. A sensor chip was sufficiently equilibrated with the running buffer and then a 5 mM nickel chloride solution was injected thereinto, so that arrangement of nickel ions was completed. Subsequently, the recombinant protein was immobilized on the sensor chip by injection of the recombinant protein solution (in the running buffer with a concentration of 100 μg/ml).

The binding reaction between the immobilized recombinant protein and human IgG was carried out as follows. Human IgG (Sigma-Aldrich Corporation) solutions were diluted and prepared to give 7 types of concentration ranging from 0.25 μg/ml to 20 μg/ml using running buffer. Each solution was injected sequentially followed by injection of the running buffer, so as to keep the solution flowing. The association and dissociation phenomena of the antibody were quantitatively observed. In addition, the flow of the solution flowing was 20 μl/min, the time for observing binding (the time for injecting an antibody solution) was 4 minutes, and the time for observing dissociation was 4 minutes. After injection of the antibody solution with each concentration and the following observation of the phenomena of association and dissociation, a 6 M guanidine hydrochloride solution was subsequently injected for 3 minutes. Thus, all human IgGs binding to the immobilized recombinant proteins were released and then regenerated using running buffer, so that they could be used for the subsequent measurements.

Changes in mass over time on the surface plasmon resonance sensor surfaces observed were measured using RU (the unit defined by Biacore) and then association rate constants (kass), dissociation rate constants (kdis), and dissociation constants (Kd=kass/kdis) were found.

[Removal of Tag Portion from Fusion Protein]

The separated and purified fusion protein (50 mg) was dissolved in 5 ml of 10 mM phosphate buffer (pH 7.0), dithiothreitol (DTT) was added therein to a final concentration of 1 mM, and the mixture was allowed to stand for 30 minutes at room temperature to reduce the cysteine residue. After the reaction, gel filtration was carried out using the PD-10 column (purchased from GE Healthcare Biosciences) to selectively recover protein portions. Thereafter, 2-nitro-5-thiocyanobenzoic acid (NTCB) was added therein to a final concentration of 5 mM, and the mixture was allowed to stand for 2 hours at room temperature to cyanate the cysteine residue. Thereafter, the resultant was dialyzed against 5 liters of 100 mM borate buffer (pH 9.5) twice for a total of 24 hours to remove NTCB and cleave the peptide chain at the cyanocysteine residue site. The reaction solution that had been subjected to the cleavage reaction simultaneously with dialysis was applied to a nickel chelate column (purchased from GE Healthcare Biosciences) to recover a portion, which did not adsorb to the column. The recovered protein sample was subjected to dialysis against 10 mM phosphate buffer (pH 7.0). After the dialysis, the target protein was dried using a centrifugal vacuum dryer. As a result of analysis using a mass spectrometer (API 150EX), the tag sequence portion was found to have been removed from the resulting modified antibody-bound protein as intended.

### [Immobilization of Recombinant Protein]

The protein from which the tag portion had been removed was dissolved to a concentration of about 4 mg/ml in 0.1 M acetate buffer (pH 4.5) containing 0.5 M NaCl to prepare a protein solution.

A protein solution (40 μl) was mixed with the commercially available NHS (N-hydroxysuccinimide)-activated sepharose carrier (20 purchased from GE Healthcare Biosciences), and the mixture was mildly stirred for about 16 hours at room temperature to perform the immobilization reaction. After the reaction, protein concentration in the solution was measured and the amount of the immobilized protein was deduced. A carrier in which an active group (i.e., N-hydroxysuccinimide) had been inactivated via treatment with ethanolamine in advance was used and a protein concentration in a solution when no protein has been immobilized was designated as the control. After the immobilization reaction, the carrier was washed with 1 ml of washing buffer (0.1 M sodium acetate, 0.5 M sodium chloride; pH 4.0). Subsequently, the carrier was mildly stirred for about 1 hour in 1 ml of inactivation buffer (0.5 M monoethanolamine, 0.5 M sodium chloride; pH 8.3) to inactivate unreacted functional groups on the carrier. The similar procedure for inactivation was repeated twice thereafter, the carrier was washed twice in 10 mM phosphate buffer (pH 7.0) containing 1 M KCl, and the carrier was then equilibrated with 10 mM phosphate buffer (pH 7.0).

### [Measurement of Igg Binding Capacity of Prepared Immobilization Carrier]

The prepared immobilization carrier (20 μl) was mixed with 1.5 mg of human-derived immunoglobulin G in 1 ml of 10 mM phosphate buffer (pH 7.0), and the mixture was mildly stirred for about 16 hours at room temperature. Thereafter, the carrier was washed 5 or more times with 1 ml of 10 mM phosphate buffer (pH 7.0) containing 1 M KCl. As a result of this procedure, no protein was detected in the final wash fluid. IgG, which had been specifically bound to the immobilization carrier, was eluted with the addition of 1 ml of 0.5 M acetic acid. The absorbance at 280 nm was measured, the amount of proteins released in 0.5 M acetic acid was determined based on the absorbance coefficient (E₂₈₀^{1%}=14.0), and the determined amount of protein was designated as the amount of the associated and dissociated and released IgG protein.

### Example 1

### Expression as Fusion Protein of Protein Containing Neither Lysine Nor Cysteine Residue

The recombinant plasmids in which the genes represented by the DNA sequences shown below had been incorporated into the BamHI-EcoRI site of the pUC18 vectors, which had been prepared by the present inventors, were used (JP Patent Application Nos. 2006-276468, 2007-057791, 2007-059175, and 2007-059204). The outline is described as follows.

[1] The recombinant plasmid, pPAA-RRRRG, is produced by incorporating the sequence shown below (SEQ ID NO: 17) into the pUC18 vector at the BamHI-EcoRI site. The sequence shown below (SEQ ID NO: 17) is a DNA sequence containing a restriction enzyme sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is a sequence derived from domain A of Staphylococcus-derived protein A which has been modified such that neither cysteine nor lysine is contained(SEQ ID NO: 2), and the R2 portion is Gly-Gly-Gly-Gly (SEQ ID NO: 3).

GGATCCTTGACAATATCTTAACTATCGTTATAATATATTGACCAGGTTAA
CTAACTAAGCAGCAAAAGGAGGAACGACTATGGCTGATAACAATTTCAAC
CGTGAACAACAAAATGCTTTCTATGAAATCTTGAATATGCCTAACTTAAA
CGAAGAACAACGCAATGGTTTCATCCAAAGCTTACGTGATGACCCAAGCC
AAAGTGCTAACCTATTGTCAGAAGCTCGTCGTTTAAATGAATCTCAAGCA
CCGGGTGGTGGCGGTGGCTGCGCTGATGACGATGACGATGACCATCATCA
CCACCATCATTAAGAATTC

The amino acid sequence of the fusion protein prepared by expressing SEQ ID NO: 17 (it is referred to as the “fusion protein PA1”) is the sequence shown below (SEQ ID NO: 18).

Ala-Asp-Asn-Asn-Phe-Asn-Arg-Glu-Gln-Gln-
Asn-Ala-Phe-Tyr-Glu-Ile-Leu-Asn-Met-Pro-
Asn-Leu-Asn-Glu-Glu-Gln-Arg-Asn-Gly-Phe-
Ile-Gln-Ser-Leu-Arg-Asp-Asp-Pro-Ser-Gln-
Ser-Ala-Asn-Leu-Leu-Ser-Glu-Ala-Arg-Arg-
Leu-Asn-Glu-Ser-Gln-Ala-Pro-Gly-Gly-Gly-
Gly-Gly-Cys-Ala-Asp-Asp-Asp-Asp-Asp-Asp-
His-His-His-His-His-His

[2] The recombinant plasmid, pPG, is produced by incorporating the sequence shown below (SEQ ID NO: 19) into the pUC18 vector at the BamHI-EcoRI site. The sequence shown below (SEQ ID NO: 19) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is a sequence derived from domain G1 of Streptococcus-derived protein G which has been modified such that neither cysteine nor lysine is contained, and the R2 portion is Gly-Gly-Gly-Gly (SEQ ID NO: 3).

GGATCCTTGACAATATCTTAACTATCTGTTATAATATATTGACCAGGTTA
ACTAACTAAGCAGCAAAAGGAGGAACGACTATGGCTTACCGTTTAATCCT
TAATGGTCGTACATTGCGTGGCGAAACAACTACTGAAGCTGTTTTGCGTG
GCGAAACAACTACTGAAGCTGTTCAATACGCTAACGACAACGGTGTTGAC
GGTGAATGGACTTACGACGATGCGACTCGTACCTTTACGGTAACTGAACG
TCCTGAGGTTATTGATGCTTCGGAGCTGACTCCTGCTGTTACTGGTGGCG
GTGGCTGCGCTGATGACGATGACGATGACCATCATCACCACCATCATTAA
GAATTC

The amino acid sequence of the fusion protein prepared by expressing SEQ ID NO: 19 (it is referred to as the “fusion protein PG1”) is the sequence shown below (SEQ ID NO: 20).

Ala-Tyr-Arg-Leu-Ile-Leu-Asn-Gly-Arg-Thr-
Leu-Arg-Gly-Glu-Thr-Thr-Thr-Glu-Ala-Val-
Asp-Ala-Ala-Thr-Ala-Glu-Arg-Val-Phe-Arg-
Gln-Tyr-Ala-Asn-Asp-Asn-Gly-Val-Asp-Gly-
Glu-Trp-Thr-Tyr-Asp-Asp-Ala-Thr-Arg-Thr-
Phe-Thr-Val-Thr-Glu-Arg-Pro-Glu-Val-Ile-
Asp-Ala-Ser-Glu-Leu-Thr-Pro-Ala-Val-Thr-
Gly-Gly-Gly-Gly-Cys-Ala-Asp-Asp-Asp-Asp-
Asp-Asp-His-His-His-His-His-His

[3] The recombinant plasmid, pPL, is produced by incorporating the sequence shown below (SEQ ID NO: 21) into the pUC18 vector at the BamHI-EcoRI site. The sequence shown below (SEQ ID NO: 21) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is a sequence derived from domain B1 of Peptostreptococcus-derived protein L which has been modified such that neither cysteine nor lysine is contained, and the R2 portion is Gly-Gly-Gly-Gly (SEQ ID NO: 3).

GGATCCTTGACAATATCTTAACTATCTGTTATAATATATTGACCAGGTTA
ACTAACTAAGCAGCAAAAGGAGGAACGACTATGGCTACTATTCGTGCTAA
TCTGATTTATGCTGATGGTCGTACTCAGACTGCTGAGTTTCGTGGTACTT
TTGAGGAGGCTACTGCTGAGGCTTATCGTTATGCTGATCTGCTGGCTCGT
GAGAATGGTCGTTATACTGTTGATGTTGCTGATCGTGGTTATACTCTGAA
TATTCGTTTTGCTGGTGGTGGCGGTGGCTGCGCTGATGACGATGACGATG
ACCATCATCACCACCATCATTAAGAATTC

The amino acid sequence of the fusion protein prepared by expressing SEQ ID NO: 21 (it is referred to as the “fusion protein PL1”) is the sequence shown below (SEQ ID NO: 22).

Ala-Thr-Ile-Arg-Ala-Asn-Leu-Ile-Tyr-Ala
Asp-Gly-Arg-Thr-Gln-Thr-Ala-Glu-Phe-Arg
Gly-Thr-Phe-Glu-Glu-Ala-Thr-Ala-Glu-Ala
Tyr-Arg-Tyr-Ala-Asp-Leu-Leu-Ala-Arg-Glu
Asn-Gly-Arg-Tyr-Thr-Val-Asp-Val-Ala-Asp
Arg-Gly-Tyr-Thr-Leu-Asn-Ile-Arg-Phe-Ala
Gly-Gly-Gly-Gly-Gly-Cys-Ala-Asp-Asp-Asp
Asp-Asp-Asp-His-His-His-His-His-His

[4] The recombinant plasmid, pAAD, is produced by incorporating the sequence shown below (SEQ ID NO: 23) into the pUC18 vector at the BamHI-EcoRI site. The sequence shown below (SEQ ID NO: 23) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is Ser-Gly-Gly-Gly-Gly (SEQ ID NO: 1), the R1 portion is 2 repeats of the sequence derived from domain A of Staphylococcus-derived protein A which has been modifies such that neither cysteine nor lysine is contained, and the R2 portion is Gly-Gly-Gly-Gly (SEQ ID NO: 3). This DNA sequence is designed in such a manner that the sequence has duplicated genes encoding the sequence portion containing neither the cysteine nor the lysine residue based on the sequence derived from domain A of protein A, the sequence contains one Cfr9I cleavage sequence (CCCGG) as a new restriction enzyme cleavage sequence, and the entire sequence can be inserted into the vector via BamHI and ExoRI cleavage.

GGATCCTTGACAATATCTTAACTATCTGTTATAATATATTGACCAGGTTA
ACTAACTAAGCAGCAAAAGGAGGAACGACTATGTCGGGCGGTGGTGGTGC
TGATAACAATTTCAACCGTGAACAACAAAATGCTTTCTATGAAATCTTGA
ATATGCCTAACTTAAACGAAGAACAACGCAATGGTTTCATCCAAAGCTTA
CGTGATGACCCAAGCCAAAGTGCTAACCTATTGTCAGAAGCTCGTCGTTT
AAATGAATCTCAAGCCCCGGGTGCTGATAACAATTTCAACCGTGAACAAC
AAAATGCTTTCTATGAAATCTTGAATATGCCTAACTTAAACGAAGAACAA
CGCAATGGTTTCATCCAAAGCTTACGTGATGACCCAAGCCAAAGTGCTAA
CCTATTGTCAGAAGCTCGTCGTTTAAATGAATCTCAAGCACCGGGTGGTG
GCGGTGGCTGCGCTGATGACGATGACGATGACCATCATCACCACCATCAT
TAAGAATTC

The amino acid sequence of the fusion protein prepared by expressing SEQ ID NO: 23 (it is referred to as the “fusion protein PA2”) is the sequence shown below (SEQ ID NO: 24).

Ser-Gly-Gly-Gly-Gly-Ala-Asp-Asn-Asn-Phe-
Asn-Arg-Glu-Gln-Gln-Asn-Ala-Phe-Tyr-Glu-
Ile-Leu-Asn-Met-Pro-Asn-Leu-Asn-Glu-Glu-
Gln-Arg-Asn-Gly-Phe-Ile-Gln-Ser-Leu-Arg-
Asp-Asp-Pro-Ser-Gln-Ser-Ala-Asn-Leu-Leu-
Ser-Glu-Ala-Arg-Arg-Leu-Asn-Glu-Ser-Leu-
Asn-Met-Pro-Asn-Leu-Asn-Glu-Glu-Gln-Arg-
Asn-Gly-Phe-Ile-Gln-Ser-Leu-Arg-Asp-Asp-
Pro-Ser-Gln-Ser-Ala-Asn-Leu-Leu-Ser-Glu-
Ala-Arg-Arg-Leu-Asn-Glu-Ser-Gln-Ala-Pro-
Gly-Gly-Gly-Gly-Gly-Cys-Ala-Asp-Asp-Asp-
Asp-Asp-Asp-His-His-His-His-His-His

[5] The recombinant plasmid, pAA3T, is produced by incorporating the sequence shown below (SEQ ID NO: 25) into the pUC18 vector at the BamHI-EcoRI site. The sequence shown below (SEQ ID NO: 25) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is Ser-Gly-Gly-Gly-Gly (SEQ ID NO: 1), the R1 portion is 3 repeats of the sequence derived from domain A of Staphylococcus-derived protein A which has been modified such that neither cysteine nor lysine is contained, and the R2 portion is Gly-Gly-Gly-Gly (SEQ ID NO: 3).

GGATCCTTGACAATATCTTAACTATCTGTTATAATATATTGACCAGGTTA
ACTAACTAAGCAGCAAAAGGAGGAACGACTATGTCGGGCGGTGGTGGTGC
TGATAACAATTTCAACCGTGAACAACAAAATGCTTTCTATGAAATCTTGA
ATATGCCTAACTTAAACGAAGAACAACGCAATGGTTTCATCCAAAGCTTA
CGTGATGACCCAAGCCAAAGTGCTAACCTATTGTCAGAAGCTCGTCGTTT
AAATGAATCTCAAGCCCCGGGTGCTGATAACAATTTCAACCGTGAACAAC
AAAATGCTTTCTATGAAATCTTGAATATGCCTAACTTAAACGAAGAACAA
CGCAATGGTTTCATCCAAAGCTTACGTGATGACCCAAGCCAAAGTGCTAA
CCTATTGTCAGAAGCTCGTCGTTTAAATGAATCTCAAGCCCCGGGTGCTG
ATAACAATTTCAACCGTGAACAACAAATGCTTTCTATGAAATCTTGAATA
TGCCTAACTTAAACGAAGAACAACGCAATGGTTTCATCCAAAGCTTACGT
GATGACCCAAGCCAAAGTGCTAACCTATTGTCAGAAGCTCGTCGTTTAAA
TGAATCTCAAGCACCGGGTGGTGGCGGTGGCTGCGCTGATGACGATGACG
ATGACCATCATCACCACCATCATTAAGAATTC

The amino acid sequence of the fusion protein prepared by expressing SEQ ID NO: (it is referred to as the “fusion protein PA3”) is the sequence shown below (SEQ ID NO: 26).

Ser-Gly-Gly-Gly-Gly-Ala-Asp-Asn-Asn-Phe-
Asn-Arg-Glu-Gln-Gln-Asn-Ala-Phe-Tyr-Glu-
Ile-Leu-Asn-Met-Pro-Asn-Leu-Asn-Glu-Glu-
Gln-Arg-Asn-Gly-Phe-Ile-Gln-Ser-Leu-Arg-
Asp-Asp-Pro-Ser-Gln-Ser-Ala-Asn-Leu-Leu-
Ser-Glu-Ala-Arg-Arg-Leu-Asn-Glu-Ser-Gln-
Ala-Pro-Gly-Ala-Asp-Asn-Asn-Phe-Asn-Arg-
Glu-Gln-Gln-Asn-Ala-Phe-Tyr-Glu-Ile-Leu-
Asn-Met-Pro-Asn-Leu-Asn-Glu-Glu-Gln-Arg-
Asn-Gly-Phe-Ile-Gln-Ser-Leu-Arg-Asp-Asp-
Pro-Ser-Gln-Ser-Ala-Asn-Leu-Leu-Ser-Glu-
Ala-Arg-Arg-Leu-Asn-Glu-Ser-Gln-Ala-Pro-
Gly-Ala-Asp-Asn-Asn-Phe-Asn-Arg-Glu-Gln-
Gln-Asn-Ala-Phe-Tyr-Glu-Ile-Leu-Asn-Met-
Pro-Asn-Leu-Asn-Glu-Glu-Gln-Arg-Asn-Gly-
Phe-Ile-Gln-Ser-Leu-Arg-Asp-Asp-Pro-Ser-
Gln-Ser-Ala-Asn-Leu-Leu-Ser-Glu-Ala-Arg-
Arg-Leu-Asn-Glu-Ser-Gln-Ala-Pro-Gly-Gly-
Gly-Gly-Gly-Cys-Ala-Asp-Asp-Asp-Asp-Asp-
Asp-His-His-His-His-His-His

[6] The recombinant plasmids produced by incorporating the DNA sequence into the pUC18 vector at the BamHI-EcoRI site, wherein the DNA sequence is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1R2 wherein the 51 portion is Ser-Gly-Gly-Gly-Gly (SEQ ID NO: 1), the R1 portion is 4 or 5 repeats of the sequence derived from domain A of Staphylococcus-derived protein A which has been modified such that neither cysteine nor lysine is not contained, and the R2 portion is Gly-Gly-Gly-Gly (SEQ ID NO: 3) were separated as pAA4Q and pAA5P.

[7] The recombinant plasmid, pGGD, is produced by incorporating the sequence shown below (SEQ ID NO: 27) into the pUC18 vector at the BamHI-EcoRI site. The sequence shown below (SEQ ID NO: 27) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the 51 portion is absent, the R1 portion is 2 repeats of the sequence derived from domain G1 of Streptococcus-derived protein G which has been modified such that neither cysteine nor lysine is contained, and the R2 portion is Gly-Gly-Gly-Gly (SEQ ID NO: 3).

GGATCCTTGACAATATCTTAACTATCTGTTATAATATATTGACCAGGTTA
ACTAACTAAGCAGCAAAAGGAGGAACGACTATGGCTTACCGTTTAATCCT
TAATGGTCGTACATTGCGTGGCGAAACAACTACTGAAGCTGTTGATGCTG
CTACTGCAGAACGTGTCTTCCGTCAATACGCTAACGACAACGGTGTTGAC
GGTGAATGGACTTACGACGATGCGACTCGTACCTTTACGGTAACTGAACG
TCCTGAGGTTATTGATGCTTCGGAGCTGACTCCTGCTGTTACTCCCGGGG
CTTACCGTTTAATCCTTAATGGTCGTACATTGCGTGGCGAAACAACTACT
GAAGCTGTTGATGCTGCTACTGCAGAACGTGTCTTCCGTCAATACGCTAA
CGACAACGGTGTTGACGGTGAATGGACTTACGACGATGCGACTCGTACCT
TTACGGTAACTGAACGTCCTGAGGTTATTGATGCTTCGGAGCTGACTCCT
GCTGTTACTGGTGGCGGTGGCTGCGCTGATGACGATGACGATGACCATCA
TCACCACCATCATTAAGAATTC

The amino acid sequence of the fusion protein prepared by expressing SEQ ID NO: 27 (it is referred to as the “fusion protein PG2”) is the sequence shown below (SEQ ID NO: 28).

Ala-Tyr-Arg-Leu-Ile-Leu-Asn-Gly-Arg-Thr-Leu-Arg-
Gly-Glu-Thr-Thr-Thr-Glu-Ala-Val-Asp-Ala-Ala-Thr-
Ala-Glu-Arg-Val-Phe-Arg-Gln-Tyr-Ala-Asn-Asp-Asn-
Gly-Val-Asp-Gly-Glu-Trp-Thr-Tyr-Asp-Asp-Ala-Thr-
Arg-Thr-Phe-Thr-Val-Thr-Glu-Arg-Pro-Glu-Val-Ile-
Asp-Ala-Ser-Glu-Leu-Thr-Pro-Ala-Val-Thr-Pro-Gly-
Ala-Tyr-Arg-Leu-Ile-Leu-Asn-Gly-Arg-Thr-Leu-Arg-
Gly-Glu-Thr-Thr-Thr-Glu-Ala-Val-Asp-Ala-Ala-Thr-
Ala-Glu-Arg-Val-Phe-Arg-Gln-Tyr-Ala-Asn-Asp-Asn-
Gly-Val-Asp-Gly-Glu-Trp-Thr-Tyr-Asp-Asp-Ala-Thr-
Arg-Thr-Phe-Thr-Val-Thr-Glu-Arg-Pro-Glu-Val-Ile-
Asp-Ala-Ser-Glu-Leu-Thr-Pro-Ala-Val-Thr-Gly-Gly-
Gly-Gly-Cys-Ala-Asp-Asp-Asp-Asp-Asp-Asp-His-His-
His-His-His-His

[8] The recombinant plasmid, pGG3T, is produced by incorporating the sequence shown below (SEQ ID NO: 29) into the pUC18 vector at the BamHI-EcoRI site. The sequence shown below (SEQ ID NO: 29) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is 3 repeats of the sequence derived from domain G1 of Streptococcus-derived protein G which has been modified such that neither cysteine nor lysine is contained, and the R2 portion is Gly-Gly-Gly-Gly (SEQ ID NO: 3).

GGATCCTTGACAATATCTTAACTATCTGTTATAATATATTGACCAGGTTA
ACTAACTAAGCAGCAAAAGGAGGAACGACTATGGCTTACCGTTTAATCCT
TAATGGTCGTACATTGCGTGGCGAAACAACTACTGAAGCTGTTGATGCTG
CTACTGCAGAACGTGTCTTCCGTCAATACGCTAACGACAACGGTGTTGAC
GGTGAATGGACTTACGACGATGCGACTCGTACCTTTACGGTAACTGAACG
TCCTGAGGTTATTGATGCTTCGGAGCTGACTCCTGCTGTTACTCCCGGGG
CTTACCGTTTAATCCTTAATGGTCGTACATTGCGTGGCGAAACAACTACT
GAAGCTGTTGATGCTGCTACTGCAGAACGTGTCTTCCGTCAATACGCTAA
CGACAACGGTGTTGACGGTGAATGGACTTACGACGATGCGACTCGTACCT
TTACGGTAACTGAACGTCCTGAGGTTATTGATGCTTCGGAGCTGACTCCT
GCTGTTACTCCCGGGGCTTACCGTTTAATCCTTAATGGTCGTACATTGCG
TGGCGAAACAACTACTGAAGCTGTTGATGCTGCTACTGCAGAACGTGTCT
TCCGTCAATACGCTAACGACAACGGTGTTGACGGTGAATGGACTTACGAC
GATGCGACTCGTACCTTTACGGTAACTGAACGTCCTGAGGTTATTGATGC
TTCGGAGCTGACTCCTGCTGTTACTGGTGGCGGTGGCTGCGCTGATGACG
ATGACGATGACCATCATCACCACCATCATTAAGAATTC

The amino acid sequence of the fusion protein prepared by expressing SEQ ID NO: 29 (it is referred to as the “fusion protein PG3”) is the sequence shown below (SEQ ID NO: 30).

Ala-Tyr-Arg-Leu-Ile-Leu-Asn-Gly-Arg-Thr-Leu-Arg-
Gly-Glu-Thr-Thr-Thr-Glu-Ala-Val-Asp-Ala-Ala-Thr-
Ala-Glu-Arg-Val-Phe-Arg-Gln-Tyr-Ala-Asn-Asp-Asn-
Gly-Val-Asp-Gly-Glu-Trp-Thr-Tyr-Asp-Asp-Ala-Thr-
Arg-Thr-Phe-Thr-Val-Thr-Glu-Arg-Pro-Glu-Val-Ile-
Asp-Ala-Ser-Glu-Leu-Thr-Pro-Ala-Val-Thr-Pro-Gly-
Ala-Tyr-Arg-Leu-Ile-Leu-Asn-Gly-Arg-Thr-Leu-Arg-
Gly-Glu-Thr-Thr-Thr-Glu-Ala-Val-Asp-Ala-Ala-Thr-
Ala-Glu-Arg-Val-Phe-Arg-Gln-Tyr-Ala-Asn-Asp-Asn-
Gly-Val-Asp-Gly-Glu-Trp-Thr-Tyr-Asp-Asp-Ala-Thr-
Arg-Thr-Phe-Thr-Val-Thr-Glu-Arg-Pro-Glu-Val-Ile-
Asp-Ala-Ser-Glu-leu-thr-Pro-Ala-Val-Thr-Pro-Gly-
Ala-Tyr-Arg-Leu-Ile-Leu-Asn-Gly-Arg-Thr-leu-Arg-
Gly-Glu-Thr-Thr-Thr-Glu-Ala-Val-Asp-Ala-Ala-Thr-
Ala-Glu-Arg-Val-phe-Arg-Gln-Tyr-Ala-Asn-Asp-Asn-
Gly-Val-Asp-Gly-Glu-Trp-Thr-Tyr-Asp-Asp-Ala-Thr-
Arg-Thr-Phe-Thr-Val-Thr-Glu-Arg-Pro-Glu-Val-Ile-
Asp-Ala-Ser-Glu-Leu-Thr-Pro-Ala-Val-Thr-Gly-Gly-
Gly-Gly-Cys-Ala-Asp-Asp-Asp-Asp-Asp-Asp-Asp-His-
His-His-His-His-His

[9] The recombinant plasmids produced by incorporating the DNA sequence into the pUC18 vector at the BamHI-EcoRI site, wherein the DNA sequence is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is 4 or 5 repeats of the sequence derived from domain G1 of Streptococcus-derived protein G which has been modified such that neither cysteine nor lysine is not contained, and the R2 portion is Gly-Gly-Gly-Gly (SEQ ID NO: 3) were separated as pGG4Q and pGG5P.

[10] The recombinant plasmid, pLLD, is produced by incorporating the sequence shown below (SEQ ID NO: 31) into the pUC18 vector at the BamHI-EcoRI site. The sequence shown below (SEQ ID NO: 31) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is 2 repeats of the sequence derived from domain B1 of Peptostreptococcus-derived protein L which has been modified such that neither cysteine nor lysine is contained, and the R2 portion is Gly-Gly-Gly-Gly (SEQ ID NO: 3).

GGATCCTTGACAATATCTTAACTATCTGTTATAATATATTGACCAGGTTA
ACTAACTAAGCAGCAAAAGGAGGAACGACTATGGCTACTATTCGTGCTAA
TCTGATTTATGCTGATGGTCGTACTCAGACTGCTGAGTTTCGTGGTACTT
TTGAGGAGGCTACTGCTGAGGCTTATCGTTATGCTGATCTGCTGCCTCGT
GAGAATGGTCGTTATACTGTTGATGTTGCTGATCGTGGTTATACTCTGAA
TATTCGTTTTGCTCCCGGGGCTACTATTCGTGCTAATCTGATTTATGCTG
ATGGTCGTACTCAGACTGCTGAGTTTCGTGGTACTTTTGAGGAGGCTACT
GCTGAGGCTTATCGTTATGCTGATCTGCTGCCTCGTGAGAATGGTCGTTA
TACTGTTGATGTTGCTGATCGTGGTTATACTCTGAATATTCGTTTTGCTG
GTGGTGGCGGTGGCTGCGCTGATGACGATGACGATGACCATCATCACCAC
CATCATTAAGAATTC

The amino acid sequence of the fusion protein prepared by expressing SEQ ID NO: 31 (it is referred to as the “fusion protein PL2”) is the sequence shown below (SEQ ID NO: 32).

Ala-Thr-Ile-Arg-Ala-Asn-Leu-Ile-Tyr-Ala-Asp-Gly-
Arg-Thr-Gln-Thr-Ala-Glu-Phe-Arg-Gly-Thr-Phe-Glu-
Glu-Ala-Thr-Ala-Glu-Ala-Tyr-Arg-Tyr-Ala-Asp-Leu-
Leu-Pro-Arg-Glu-Asn-Gly-Arg-Tyr-Thr-Val-Asp-Val-
Ala-Asp-Arg-Gly-Tyr-Thr-Leu-Asn-Ile-Arg-Phe-Ala-
Pro-Gly-Ala-Thr-Ile-Arg-Ala-Asn-Leu-Ile-Tyr-Ala-
Asp-Gly-Arg-Thr-Gln-Thr-Ala-Glu-Phe-Arg-Gly-Thr-
Phe-Glu-Glu-Ala-Thr-Ala-Glu-Ala-Tyr-Agr-Tyr-Ala-
Asp-Leu-leu-Pro-Arg-Glu-Asn-Gly-Arg-Tyr-Thr-Val-
Asp-Val-Ala-Asp-Arg-Gly-Tyr-Thr-Leu-Asn-Ile-Arg-
Phe-Ala-Gly-Gly-Gly-Gly-Gly-Cys-Ala-Asp-Asp-Asp-
Asp-Asp-Asp-His-His-His-His-His-His

[11] The recombinant plasmid, pLL3T, is produced by incorporating the sequence shown below (SEQ ID NO: 33) into the pUC18 vector at the BamHI-EcoRI site. The sequence shown below (SEQ ID NO: 33) is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is 3 repeats of the sequence derived from domain B1 of Peptostreptococcus-derived protein L which has been modified such that neither cysteine nor lysine is contained, and the R2 portion is Gly-Gly-Gly-Gly (SEQ ID NO: 3).

GGATCCTTGACAATATCTTAACTATCTGTTATAATATATTGACCAGGTTA
ACTAACTAAGCAGCAAAAGGAGGAACGACTATGGCTACTATTCGTGCTAA
TCTGATTTATGCTGATGGTCGTACTCAGACTGCTGAGTTTCGTGGTACTT
TTGAGGAGGCTACTGCTGAGGCTTATCGTTATGCTGATCTGCTGCCTCGT
GAGAATGGTCGTTATACTGTTGATGTTGCTGATCGTGGTTATACTCTGAA
TATTCGTTTTGCTCCCGGGGCTACTATTCGTGCTAATCTGATTTATGCTG
ATGGTCGTACTCAGACTGCTGAGTTTCGTGGTACTTTTGAGGAGGCTACT
GCTGAGGCTTATCGTTATGCTGATCTGCTGCCTCGTGAGAATGGTCGTTA
TACTGTTGATGTTGCTGATCGTGGTTATACTCTGAATATTCGTTTTGCTC
CCGGGGCTACTATTCGTGCTAATCTGATTTATGCTGATGGTCGTACTCAG
ACTGCTGAGTTTCGTGGTACTTTTGAGGAGGCTACTGCTGAGGCTTATCG
TTATGCTGATCTGCTGCCTCGTGAGAATGGTCGTTATACTGTTGATGTTG
CTGATCGTGGTTATACTCTGAATATTCGTTTTGCTGGTGGTGGCGGTGGC
TGCGCTGATGACGATGACGATGACCATCATCACCACCATCATTAAGAATT
C

The amino acid sequence of the fusion protein prepared by expressing SEQ ID NO: 33 (it is referred to as the “fusion protein PL3”) is the sequence shown below (SEQ ID NO: 34).

Ala-Thr-Ile-Arg-Ala-Asn-Leu-Ile-Tyr-Ala-Asp-Gly-
Arg-Thr-Gln-Thr-Ala-Glu-Phe-Arg-Gly-Thr-Phe-Glu-
Glu-Ala-Thr-Ala-Glu-Ala-Tyr-Arg-Tyr-Ala-Asp-Leu-
Leu-Pro-Arg-Glu-Asn-Gly-Arg-Tyr-Thr-Val-Asp-Val-
Ala-Asp-Arg-Gly-Tyr-Thr-Leu-Asn-Ile-Arg-Phe-Ala-
Pro-Gly-Ala-Thr-Ile-Arg-Ala-Asn-Leu-Ile-Tyr-Ala-
Asp-Gly-Arg-Thr-Gln-Thr-Ala-Glu-Phe-Arg-Gly-Thr-
Phe-Glu-Glu-Ala-Thr-Ala-Glu-Ala-Tyr-Arg-Tyr-Ala-
Asp-Leu-Leu-Pro-Arg-Glu-Asn-Gly-Arg-Tyr-Thr-Val-
Asp-Val-Ala-Asp-Arg-Gly-Tyr-Thr-Leu-Asn-Ile-Arg-
Phe-Ala-Pro-Gly-Ala-Thr-Ile-Arg-Ala-Asn-Leu-Ile-
Tyr-Ala-Asp-Gly-Arg-Thr-Gln-Thr-Ala-Glu-Phe-Arg-
Gly-Thr-Phe-Glu-Glu-Ala-Thr-Ala-Glu-Ala-Tyr-Arg-
Tyr-Ala-Asp-Leu-Leu-Pro-Arg-Glu-Asn-Gly-Arg-Tyr-
Thr-Val-Asp-Val-Ala-Asp-Arg-Gly-Tyr-Thr-Leu-Asn-
Ile-Arg-Phe-Ala-Gly-Gly-Gly-Gly-Gly-Cys-Ala-Asp-
Asp-Asp-Asp-Asp-Asp-His-His-His-His-His-His

[12] The recombinant plasmids produced by incorporating the DNA sequence into the pUC18 vector at the BamHI-EcoRI site, wherein the DNA sequence is a DNA sequence capable of expressing the amino acid sequence wherein Cys-Ala as the C1 portion and Asp-Asp-Asp-Asp-Asp-Asp-His-His-His-His-His-His (SEQ ID NO: 16) which are sequences for cleavage and tag purification as the T1 portion are fused to carboxy terminal side of the protein sequence represented by the general formula S1-R1-R2 wherein the S1 portion is absent, the R1 portion is 4 or 5 repeats of the sequence derived from domain B1 of Peptostreptococcus-derived protein L which has been modified such that neither cysteine nor lysine is not contained, and the R2 portion is Gly-Gly-Gly-Gly (SEQ ID NO: 3) were prepared.

### Example 2

### Expression of Fusion Protein in E. coli and Separation and Purification Thereof

As the recombinant plasmids described in Example 1, E. coli JM109 strains in which pPAA-RRRRG pAAD, pAA3T, pPG, pGGD, pGG3T, pPL, pLLD, and pLL3T had been incorporated were cultured, and the proteins were separated and purified from the cell-free extract of the disrupted cultured cells using the nickel chelate column (purchased from GE Healthcare Biosciences). This procedure was carried out by the method described above. Proteins obtained via purification and separation are designated as PA1, PA2, PA3, PG1, PG2, PG3, PL1, PL2, and PL3, and the yields thereof are as shown in Table 1 (mg/2 l of culture).

TABLE 1
Yield of purified fusion proteins

| Recombinant plasmid | Protein | Amount of purified protein (mg/2 l) |
|---|---|---|
| pPP-RRRRG | PA1 | 110 |
| pAAD | PA2 | 198 |
| pAA3T | PA3 | 190 |
| pPG | PG1 | 356 |
| pGGD | PG2 | 59 |
| pGG3T | PG3 | 12 |
| pPL | PL1 | 63 |
| pLLD | PL2 | 13 |
| pLL3T | PL3 | 5 |

The binding properties of fusion proteins obtained via purification to the human polyclonal IgG were measured using the Biacore system, and the results are shown in Table 2.

TABLE 2
Antibody-binding properties of fusion proteins

| Protein | Kass [M⁻¹s⁻¹] × 10⁻⁵ | Koff [s⁻¹] × 10⁵ | Kd [M] × 10¹⁰ |
|---|---|---|---|
| PA1 | 1.84 | 11.76 | 6.34 |
| PA2 | 5.75 | 18.3 | 3.18 |
| PA3 | 7.86 | 13.3 | 1.69 |
| PG1 | 4.01 | 15.4 | 3.84 |
| PG2 | 8.64 | 10.0 | 1.15 |
| PG3 | 11.2 | 7.63 | 0.68 |
| PL1 | 1.51 | 31.2 | 20.6 |
| PL2 | 2.46 | 26.4 | 13.4 |
| PL3 | 3.01 | 23.7 | 7.88 |

As is apparent from the results shown in Table 2, the R1 portion exerting the functions and containing neither the cysteine nor lysine residues maintains the original functions, i.e., the binding ability to the human polyclonal IgG.

### Example 3

### Removal of Tag Sequence Portion from Fusion Protein

Fusion protein of the separated and purified PA1, PA2, PA3, PG1, PG2, and PL1 (50 mg each) were subjected to the cleavage and removal of the tag portion sequence utilizing the cyanocysteine reaction. Proteins that did not bind to the nickel chelate column (purchased from GE Healthcare Biosciences) were separated. Products other than those cleaved by the cyanocysteine reaction had His-tags. This indicates that all the recovered proteins are proteins represented by the general formula S1-R1-R2. The recovered proteins corresponding to the original fusion protein were designated as PAD1, PAD2, PAD3, PGD1, PGD2, and PLD1, respectively. The yields thereof are shown in Table 3. Proteins containing neither the cysteine nor lysine residues were prepared with a recovery rate of approximately 60% or more.

TABLE 3
Yield of protein after removal of tag sequence portion
(from 50 mg of fusion protein)

| Recombinant plasmid | Protein | Amount of purified protein (mg) |
|---|---|---|
| PA1 | PAD1 | 31 |
| PA2 | PAD2 | 33 |
| PA3 | PAD3 | 35 |
| PG1 | PGD1 | 28 |
| PG2 | PGD2 | 30 |
| PL1 | PLD1 | 26 |

### Example 4

### Immobilization of Protein Utilizing Amino Terminal Amino Group

The 6 types of proteins prepared in Example 3 were dissolved at concentrations of about 4 mg/ml in 0.1 M acetate buffer (pH 4.5) containing 0.5M NaCl to prepare a protein solution. The thus-prepared protein solution (40 μl) was mixed with 20 μl of the NHS (N-hydroxysuccinimide)-activated sepharose carrier (purchased from GE Healthcare Biosciences), the mixture was mildly stirred for about 16 hours at room temperature, and the protein concentrations in the solution were measured. As a result, all the protein concentrations were found to be 0.1 mg/ml or lower. This demonstrates that proteins were substantially quantitatively immobilized under the above conditions. This indicates that a carrier on which proteins are immobilized at about 8 mg/ml of the carrier is prepared under the above conditions.

PAD1 proteins were immobilized by increasing the concentrations to 10 mg/ml, 20 mg/ml, 30 mg/ml, and 40 mg/ml. As a result, a tendency of saturation at concentrations of 20 mg/ml or higher was observed as shown in Table 4. In the case of the NHS (N-hydroxysuccinimide)-activated sepharose carrier (purchased from GE Healthcare Biosciences), a possibility of immobilization of up to about 40 mg/ml of PAD1 was found.

TABLE 4
Dependence of amount immobilized on
amount of protein introduced

| | Amount immobilized |
|---|---|
| Protein concentration (mg/ml) | (mg/0.02 ml of carrier) |
| 4 | 0.16 |
| 10 | 0.40 |
| 20 | 0.65 |
| 30 | 0.78 |
| 40 | 0.82 |

### Example 5

### Binding Capacity of Human Polyclonal IgG Immobilized on Immobilization Carrier in Orientation-Controlled Manner at a Single Amino Terminus

In accordance with Example 4, immobilization carriers on which substantially the maximal amounts of PAD1, PAD2, and PAD3 were immobilized were prepared. With the use of 20 μl each of the prepared carriers, the binding capacity of human polyclonal IgG was measured. Human polyclonal IgG was mixed in 10 mM phosphate buffer (pH 7.0), the resultant was mildly stirred for about 16 hours at room temperature to allow antibody molecules to bind to the carriers, proteins that were nonspecifically adsorbed were removed with the use of 10 mM phosphate buffer (pH 7.0) containing 1 M KCl, and the amount of antibody proteins released in a 0.5 M acetic acid solution was measured as the amount of binding.

The binding capacities of human polyclonal IgG when PAD1, PAD2, and PAD3 were immobilized were found to be high as shown in Table 5.

TABLE 5
Antibody binding shown by immobilization carrier

| | Number of | Amount of antibody binding |
|---|---|---|
| Protein immobilized | binding domains | (mg/ml of carrier) |
| PAD1 | 1 | 39 |
| PAD2 | 2 | 50 |
| PAD3 | 3 | 63 |

1. An immobilized protein bound to an immobilization carrier at a protein amino terminus via the sole α-amino group of the protein consisting of an amino acid sequence containing neither lysine residues nor cysteine residues represented by the general formula S1-R1-R2, wherein:
the sequences are oriented from the amino terminal side to the carboxy terminal side;
the sequence of the S1 portion may be absent, but when the sequence of the S1 portion is present, the sequence of the S1 portion is a spacer sequence composed of amino acid residues other than lysine and cysteine residues;
the sequence of the R1 portion is the sequence of a subject protein to be immobilized and contains neither lysine residues nor cysteine residues; and
the sequence of the R2 portion may be absent, but when the sequence of the R2 portion is present, the sequence of the R2 portion is a spacer sequence composed of amino acid residues other than lysine and cysteine residues.

2. The immobilized protein according to claim 1 consisting of the amino acid sequence represented by the general formula S1-R1-R2, wherein, in the amino acid sequence of the general formula S1-R1-R2, the sequence of the R1 portion is:
the sequence remaining unchanged when the amino acid sequence of a naturally derived protein contains neither lysine residues nor cysteine residues; or
the amino acid sequence of a protein that consists of an amino acid sequence modified to contain neither lysine residues nor cysteine residues and has functions equivalent to those of a naturally derived protein in which a modified amino acid sequence is obtained by substituting all lysine and cysteine residues in the amino acid sequence with amino acid residues other than lysine and cysteine residues, when the sequence contains lysine residues and cysteine residues.

3. The immobilized protein according to claim 1 wherein, in the amino acid sequence of the general formula S1-R1-R2, the sequence of the R1 portion has a function of interacting specifically with an antibody molecule.

4. The immobilized protein according to claim 3 wherein, in the amino acid sequence represented by the general formula S1-R1-R2,
S1=Ser-Gly-Gly-Gly-Gly or is absent,
R1=(Ala-Asp-Asn-Asn-Phe-Asn-Arg-Glu-Gln-Gln- Asn-Ala-Phe-Tyr-Glu-Ile-Leu-Asn-Met-Pro- Asn-Leu-Asn-Glu-Glu-Gln-Arg-Asn-Gly-Phe- Ile-Gln-Ser-Leu-Arg-Asp-Asp-Pro-Ser-Gln- Ser-Ala-Asn-Leu-Leu-Ser-Glu-Ala-Arg-Arg- Leu-Asn-Glu-Ser-Gln-Ala-Pro-Gly)n (where n is an arbitrary integer ranging from 1 to 5), and
R2=Gly-Gly-Gly-Gly or is absent.

5. The immobilized protein according to claim 3 wherein, in the amino acid sequence represented by the general formula S1-R1-R2,
S1=absent;
R1=(Ala-Tyr-Arg-Leu-Ile-Leu-Asn-Gly-Arg-Thr- Leu-Arg-Gly-Glu-Thr-Thr-Thr-Glu-Ala-Val- Asp-Ala-Ala-Thr-Ala-Glu-Arg-Val-Phe-Arg- Gln-Tyr-Ala-Asn-Asp-Asn-Gly-Val-Asp-Gly- Glu-Trp-Thr-Tyr-Asp-Asp-Ala-Thr-Arg-Thr- Phe-Thr-Val-Thr-Glu-Arg-Pro-Glu-Val-Ile- Asp-Ala-Ser-Glu-Leu-Thr-Pro-Ala-Val-Thr-Pro-Gly)n (where n is an arbitrary integer ranging from 1 to 5), and
R2=Gly-Gly-Gly-Gly or is absent.

6. A carrier on which the immobilized proteins according to claim 1 are immobilized.

7. The immobilized protein according to claim 2 wherein, in the amino add sequence of the general formula S1-R1-R2, the sequence of the R1 portion has a function of interacting specifically with an antibody molecule.

8. The immobilized protein according to claim 7 wherein, in the amino acid sequence represented by the general formula S1-R1-R2,
S1=Ser-Gly-Gly-Gly-Gly or is absent,
R1=(Ala-Asp-Asn-Asn-Phe-Asn-Arg-Glu-Gln-Gln- Asn-Ala-Phe-Tyr-Glu-Ile-Leu-Asn-Met-Pro- Asn-Leu-Asn-Glu-Glu-Gln-Arg-Asn-Gly-Phe- Ile-Gln-Ser-Leu-Arg-Asp-Asp-Pro-Ser-Gln- Ser-Ala-Asn-Leu-Leu-Ser-Glu-Ala-Arg-Arg- Leu-Asn-Glu-Ser-Gln-Ala-Pro-Gly)n (where n is an arbitrary integer ranging from 1 to 5), and
R2=Gly-Gly-Gly-Gly or is absent.

9. The immobilized protein according to claim 7 wherein, in the amino acid sequence represented by the general formula S1-R1-R2,
S1=absent;
R1=(Ala-Tyr-Arg-Leu-Ile-Leu-Asn-Gly-Arg-Thr- Leu-Arg-Gly-Glu-Thr-Thr-Thr-Glu-Ala-Val- Asp-Ala-Ala-Thr-Ala-Glu-Arg-Val-Phe-Arg- Gln-Tyr-Ala-Asn-Asp-Asn-Gly-Val-Asp-Gly- Glu-Trp-Thr-Tyr-Asp-Asp-Ala-Thr-Arg-Thr- Phe-Thr-Val-Thr-Glu-Arg-Pro-Glu-Val-Ile- Asp-Ala-Ser-Glu-Leu-Thr-Pro-Ala-Val-Thr-Pro-Gly)n (where n is an arbitrary integer ranging from 1 to 5), and
R2=Gly-Gly-Gly-Gly or is absent.

10. A carrier on which the immobilized proteins according to claim 2 are immobilized.

11. A carrier on which the immobilized proteins according to claim 3 are immobilized.

12. A carrier on which the immobilized proteins according to claim 4 are immobilized.

13. A carrier on which the immobilized proteins according to claim 5 are immobilized.

14. A carrier on which the immobilized proteins according to claim 6 are immobilized.

15. A carrier on which the immobilized proteins according to claim 7 are immobilized.

16. A carrier on which the immobilized proteins according to claim 8 are immobilized.

17. A carrier on which the immobilized proteins according to claim 9 are immobilized.


SEQUENCE LISTING (34 sequences)

SEQ ID NO: 1 - protein, 5 aa, Artificial
  Synthetic
SGGGG

SEQ ID NO: 2 - protein, 58 aa, Artificial
  Synthetic
ADNNFNREQQNAFYEILNMPNLNEEQRNGFIQSLRDDPSQSANLLSEARRLNESQAPG

SEQ ID NO: 3 - protein, 4 aa, Artificial
  Synthetic
GGGG

SEQ ID NO: 4 - protein, 72 aa, Artificial
  Synthetic
AYRLILNGRTLRGETTTEAVDAATAERVFRQYANDNGVDGEWTYDDATRTFTVTERPEVIDASELTPAVTPG

SEQ ID NO: 5 - protein, 62 aa, Artificial
  Synthetic
ATIRANLIYADGRTQTAEFRGTFEEATAEAYRYADLLARENGRYTVDVADRGYTLNIRFAPG

SEQ ID NO: 6 - protein, 58 aa, Staphylococcus sp.
ADNNFNKEQQNAFYEILNMPNLNEEQRNGFIQSLKDDPSQSANLLSEAKKLNESQAPK

SEQ ID NO: 7 - protein, 58 aa, Artificial; identical to SEQ ID NO: 2
  Synthetic

SEQ ID NO: 8 - protein, 70 aa, Streptococcus sp.
TYKLILNGKTLKGETTTEAVDAATAEKVFKQYANDNGVDGEWTYDDATKTFTVTERPEVIDASELTPAVT

SEQ ID NO: 9 - protein, 72 aa, Artificial; identical to SEQ ID NO: 4
  Synthetic

SEQ ID NO: 10 - protein, 60 aa, Peptostreptococcus sp.
VTIKANLIYADGKTQTAEFKGTFEEATAEAYRYADLLAKENGKYTVDVADKGYTLNIKFA

SEQ ID NO: 11 - protein, 62 aa, Artificial; identical to SEQ ID NO: 5
  Synthetic

SEQ ID NO: 12 - protein, 6 aa, Artificial
  Synthetic
HQHQHQ

SEQ ID NO: 13 - protein, 10 aa, Artificial
  Synthetic
EQKLISEEDL

SEQ ID NO: 14 - protein, 9 aa, Artificial
  Synthetic
YPYDVPDYA

SEQ ID NO: 15 - protein, 8 aa, Artificial
  Synthetic
DYKDDDDK

SEQ ID NO: 16 - protein, 12 aa, Artificial
  Synthetic
DDDDDDHHHHHH

SEQ ID NO: 17 - DNA, 320 bp, Artificial
  Synthetic
ggatccttgacaatatcttaactatctgttataatatattgaccaggttaactaactaagcagcaaaaggaggaacgactatggctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagcaccgggtggtggcggtggctgcgctgatgacgatgacgatgaccatcatcaccaccatcattaagaattc

SEQ ID NO: 18 - protein, 76 aa, Artificial
  Synthetic
ADNNFNREQQNAFYEILNMPNLNEEQRNGFIQSLRDDPSQSANLLSEARRLNESQAPGGGGGCADDDDDDHHHHHH

SEQ ID NO: 19 - DNA, 356 bp, Artificial
  Synthetic
ggatccttgacaatatcttaactatctgttataatatattgaccaggttaactaactaagcagcaaaaggaggaacgactatggcttaccgtttaatccttaatggtcgtacattgcgtggcgaaacaactactgaagctgttttgcgtggcgaaacaactactgaagctgttcaatacgctaacgacaacggtgttgacggtgaatggacttacgacgatgcgactcgtacctttacggtaactgaacgtcctgaggttattgatgcttcggagctgactcctgctgttactggtggcggtggctgcgctgatgacgatgacgatgaccatcatcaccaccatcattaagaattc

SEQ ID NO: 20 - protein, 88 aa, Artificial
  Synthetic
AYRLILNGRTLRGETTTEAVDAATAERVFRQYANDNGVDGEWTYDDATRTFTVTERPEVIDASELTPAVTGGGGCADDDDDDHHHHHH

SEQ ID NO: 21 - DNA, 329 bp, Artificial
  Synthetic
ggatccttgacaatatcttaactatctgttataatatattgaccaggttaactaactaagcagcaaaaggaggaacgactatggctactattcgtgctaatctgatttatgctgatggtcgtactcagactgctgagtttcgtggtacttttgaggaggctactgctgaggcttatcgttatgctgatctgctggctcgtgagaatggtcgttatactgttgatgttgctgatcgtggttatactctgaatattcgttttgctggtggtggcggtggctgcgctgatgacgatgacgatgaccatcatcaccaccatcattaagaattc

SEQ ID NO: 22 - protein, 79 aa, Artificial
  Synthetic
ATIRANLIYADGRTQTAEFRGTFEEATAEAYRYADLLARENGRYTVDVADRGYTLNIRFAGGGGGCADDDDDDHHHHHH

SEQ ID NO: 23 - DNA, 509 bp, Artificial
  Synthetic
ggatccttgacaatatcttaactatctgttataatatattgaccaggttaactaactaagcagcaaaaggaggaacgactatgtcgggcggtggtggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagccccgggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagcaccgggtggtggcggtggctgcgctgatgacgatgacgatgaccatcatcaccaccatcattaagaattc

SEQ ID NO: 24 - protein, 119 aa, Artificial
  Synthetic
SGGGGADNNFNREQQNAFYEILNMPNLNEEQRNGFIQSLRDDPSQSANLLSEARRLNESLNMPNLNEEQRNGFIQSLRDDPSQSANLLSEARRLNESQAPGGGGGCADDDDDDHHHHHH

SEQ ID NO: 25 - DNA, 683 bp, Artificial
  Synthetic
ggatccttgacaatatcttaactatctgttataatatattgaccaggttaactaactaagcagcaaaaggaggaacgactatgtcgggcggtggtggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagccccgggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagccccgggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagcaccgggtggtggcggtggctgcgctgatgacgatgacgatgaccatcatcaccaccatcattaagaattc

SEQ ID NO: 26 - protein, 197 aa, Artificial
  Synthetic
SGGGGADNNFNREQQNAFYEILNMPNLNEEQRNGFIQSLRDDPSQSANLLSEARRLNESQAPGADNNFNREQQNAFYEILNMPNLNEEQRNGFIQSLRDDPSQSANLLSEARRLNESQAPGADNNFNREQQNAFYEILNMPNLNEEQRNGFIQSLRDDPSQSANLLSEARRLNESQAPGGGGGCADDDDDDHHHHHH

SEQ ID NO: 27 - DNA, 572 bp, Artificial
  Synthetic
ggatccttgacaatatcttaactatctgttataatatattgaccaggttaactaactaagcagcaaaaggaggaacgactatggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactcccggggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactggtggcggtggctgcgctgatgacgatgacgatgaccatcatcaccaccatcattaagaattc

SEQ ID NO: 28 - protein, 160 aa, Artificial
  Synthetic
AYRLILNGRTLRGETTTEAVDAATAERVFRQYANDNGVDGEWTYDDATRTFTVTERPEVIDASELTPAVTPGAYRLILNGRTLRGETTTEAVDAATAERVFRQYANDNGVDGEWTYDDATRTFTVTERPEVIDASELTPAVTGGGGCADDDDDDHHHHHH

SEQ ID NO: 29 - DNA, 788 bp, Artificial
  Synthetic
ggatccttgacaatatcttaactatctgttataatatattgaccaggttaactaactaagcagcaaaaggaggaacgactatggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactcccggggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactcccggggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactggtggcggtggctgcgctgatgacgatgacgatgaccatcatcaccaccatcattaagaattc

SEQ ID NO: 30 - protein, 232 aa, Artificial
  Synthetic
AYRLILNGRTLRGETTTEAVDAATAERVFRQYANDNGVDGEWTYDDATRTFTVTERPEVIDASELTPAVTPGAYRLILNGRTLRGETTTEAVDAATAERVFRQYANDNGVDGEWTYDDATRTFTVTERPEVIDASELTPAVTPGAYRLILNGRTLRGETTTEAVDAATAERVFRQYANDNGVDGEWTYDDATRTFTVTERPEVIDASELTPAVTGGGGCADDDDDDHHHHHH

SEQ ID NO: 31 - DNA, 515 bp, Artificial
  Synthetic
ggatccttgacaatatcttaactatctgttataatatattgaccaggttaactaactaagcagcaaaaggaggaacgactatggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctcccggggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctggtggtggcggtggctgcgctgatgacgatgacgatgaccatcatcaccaccatcattaagaattc

SEQ ID NO: 32 - protein, 141 aa, Artificial
  Synthetic
ATIRANLIYADGRTQTAEFRGTFEEATAEAYRYADLLPRENGRYTVDVADRGYTLNIRFAPGATIRANLIYADGRTQTAEFRGTFEEATAEAYRYADLLPRENGRYTVDVADRGYTLNIRFAGGGGGCADDDDDDHHHHHH

SEQ ID NO: 33 - DNA, 701 bp, Artificial
  Synthetic
ggatccttgacaatatcttaactatctgttataatatattgaccaggttaactaactaagcagcaaaaggaggaacgactatggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctcccggggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctcccggggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctggtggtggcggtggctgcgctgatgacgatgacgatgaccatcatcaccaccatcattaagaattc

SEQ ID NO: 34 - protein, 203 aa, Artificial
  Synthetic
ATIRANLIYADGRTQTAEFRGTFEEATAEAYRYADLLPRENGRYTVDVADRGYTLNIRFAPGATIRANLIYADGRTQTAEFRGTFEEATAEAYRYADLLPRENGRYTVDVADRGYTLNIRFAPGATIRANLIYADGRTQTAEFRGTFEEATAEAYRYADLLPRENGRYTVDVADRGYTLNIRFAGGGGGCADDDDDDHHHHHH